(19) United States
(12) Patent Application Publication
(10) Pub. No.: US 2021/0378623 A1
(43) Pub. Date: Dec. 9, 2021

(54) Control System For X-Ray Imaging System

(71) Applicants: Haim MELMAN, Kfar Saba (IL); CONTROLLRAD SYSTEMS INC., Radnor, PA (US)

(72) Inventors: Haim Melman, Kfar Saba (IL); Guillaume Bailliard, Duluth, GA (US)

(21) Appl. No.: 17/286,830

(22) PCT Filed: Oct. 20, 2019
(86) PCT No.: PCT/IB2019/058931

§ 371 Date: Apr. 20, 2021

Related U.S. Application Data
(60) Provisional application No. 62/748,540, filed on Oct. 22, 2018.

(51) Int. Cl. A61B 6/00; A61B 6/04

(52) U.S. Cl. CPC A61B 6/545; A61B 6/0407; A61B 6/4035; A61B 6/405; A61B 6/547; A61B 6/542; A61B 6/4441

(57) ABSTRACT

An x-ray system comprising: at least two system statuses; at least one system status value associated with at least one of the at least two system statuses; at least one trigger; at least one activation parameter associated with the at least one trigger; the at least one activation parameter is selected from the group consisting of: (a) at least one system status value; (b) at least one system status value with a tolerance; and (c) at least one range of system status values; at least one set associated with the at least one trigger, the at least one set comprises at least one system status parameter, the at least one system status parameter comprises at least one of: (1) a system status value; (2) a system status value with a tolerance; and (3) a range of system status values; and wherein the system status is changed according to the set upon activation of the at least one trigger.

### FIELD OF THE INVENTION

The present invention generally relates to the field of x-ray imaging and more particularly to the field of controlling motion of filters and x-ray systems in x-ray imaging systems.

### CROSS-REFERENCE TO RELATED PATENT APPLICATIONS

This patent application claims priority from and is related to U.S. Provisional Patent Application Ser. No. 62/748,540, filed Oct. 22, 2018, this U.S. Provisional Patent Application incorporated by reference in its entirety herein.

### BACKGROUND OF THE INVENTION

A typical x-ray imaging system can be moved relative to a patient to set the imaging geometry. In some x-ray systems this is done by an operator moving the x-ray system by hand. In other x-ray systems the motion is based on actuators (typically motors with suitable transmission mechanism) and electrical switches to control the motion of the x-ray system.

X-ray systems also include collimators that are designed to block radiation outside a Region of Interest (ROI) that is typically selected to be smaller than the available field of View (FOV), in accordance to the size of the relevant anatomy of the patient. Some collimators are moved by an operator moving the collimator by hand. In other collimators the motion is based on actuators (typically motors with suitable transmission mechanism) and electrical switches to control the motion of the collimators.

Readjustment of the x-ray system orientation and collimator position is a time consuming process and requires the operator's attention and verification of the final settings. In many medical procedures there are x-ray system and collimator positions that are used repeatedly. Repeating these settings repeatedly consumes time and, in some medical applications, are destructive.

It is desired to provide an apparatus and method that will automate part or all these adjustments in order to save time during x-ray imaging procedures and release the operator from such processes.

### SUMMARY

According to an aspect of the present invention there is provided an x-ray system comprising: an x-ray source; a detector; a table; at least one moving part configured to at least one of: a. position the x-ray source relative to a patient; b. position the detector relative to a patient; and c. position the table; at least one system position status value associated with at least one system position status of the at least one moving part; a filter; means for automatically setting the filter according to the at least one system position status; the means for automatically setting the filter according to the at least one system position status comprises: at least one trigger; and at least one activation parameter associated with the at least one trigger; the at least one activation parameter is selected from the group consisting of: (a) at least one system position status value; (b) at least one system position status value with a tolerance; and (c) at least one range of system position status values; at least one set associated with the at least one trigger, the at least one set comprises at least one filter status parameter, the at least one filter status parameter comprises at least one of: (1) a filter status value of the filter; (2) a filter status value with a tolerance; and (3) a range of filter status values; and wherein the filter status is changed according to the set upon activation of the at least one trigger.

A user may configure at least one of: (a) at least one activation parameter of the at least one trigger; and (b) at least one filter status parameter of the at least one set.

The at least one system position status value may be provided from at least one of the group consisting of: (a) at least one accelerometer connected with at least one moving part of the x-ray system; (b) at least one encoder indicating position of at least one moving part of the x-ray system; (c) a camera; (d) analysis of at least one x-ray image; (e) analysis of at least one x-ray image comprising fiducials of known geometry; (f) analysis of images from at least one camera attached to at least one moving part of the x-ray system; and (g) analysis of images from at least one camera configured to acquire images of at least one moving part of the x-ray system.

The system may further be configured to reduce radiation during motion of at least one of the at least one moving part.

The radiation reduction may be performed according to one of the trigger and a second trigger.

A user may configure at least one of: (a) an activation motion parameter of the at least one trigger; and (b) at least one radiation reduction parameter included in the at least one set associated with the at least one trigger.

The motion status information may be provided from at least one of the group consisting of: (a) at least one accelerometer connected to the at least one moving part; (b) at least one encoder indicating position of at least one of the at least one moving parts; (c) a camera; (d) x-ray image analysis; (e) analyzing x-ray image that includes fiducials of known geometry; and (f) analyzing images from at least one camera attached to at least one of the moving parts; and (g) analyzing images from at least one camera configured to acquire images of at least one of the moving part.

The system may further comprise means for enhancing at least one image obtained with the reduced radiation.

The enhancement of at least one image obtained with the reduced radiation may comprise increasing brightness of at least a part of the image.

According to another aspect of the present invention there is provided an x-ray system comprising: at least two system statuses; at least one system status value associated with at least one of the at least two system statuses; at least one trigger; at least one activation parameter associated with the at least one trigger; the at least one activation parameter is selected from the group consisting of: (a) at least one system status value; (b) at least one system status value with a tolerance; and (c) at least one range of system status values; at least one set associated with the at least one trigger, the at least one set comprises at least one system status parameter, the at least one system status parameter comprises at least one of: (1) a system status value; (2) a system status value with a tolerance; and (3) a range of system status values; and wherein the system status is changed according to the set upon activation of the at least one trigger.

The at least one activation parameter associated with the trigger may be specified by a user.

The at least one system status parameter of the at least one set may be specified by a user.

The at least one system status parameter of the at least one set may be a status parameter of a filter.

The at least one set may comprise at least one radiation reduction status parameter.

The at least one set may comprise at least one x-ray tube voltage parameter.

The at least one set may comprise at least one x-ray tube current parameter.

The at least one set may comprise at least one x-ray system position status parameter.

The x-ray system may be configured to automatically move to the at least one x-ray system position of the at least one set upon activation of the at least one trigger.

At least one datum of at least one of the at least two system statuses may be provided from at least one of the group consisting of: (a) at least one accelerometer connected with at least one moving part of the x-ray system; (b) at least one encoder indicating position of at least one moving part of the x-ray system; (c) a camera; (d) analysis of at least one x-ray image; (e) analysis of at least one x-ray image comprising fiducials of known geometry; (f) analysis of images from at least one camera attached to at least one moving part of the x-ray system; and (g) analysis of images from at least one camera configured to acquire images of at least one moving part of the x-ray system.

The at least one trigger may be selected from the group consisting of: (1) a mechanical switch; (2) an electrical switch; (3) a user interface graphical switch displayed by a computer on a monitor.

The at least one set may comprise at least one parameter related to image processing.

The at least one activation parameter may comprise at least one alpha-numeric string.

The at least one activation parameter may comprise at least one motion system status.

At least one datum of the at least one motion system status may be provided from at least one of the group consisting of: (a) at least one accelerometer connected with at least one moving part of the x-ray system; (b) at least one encoder indicating position of at least one moving part of the x-ray system; (c) a camera; (d) analysis of at least one x-ray image; (e) analysis of at least one x-ray image comprising fiducials of known geometry; (f) analysis of images from at least one camera attached to at least one moving part of the x-ray system; and (g) analysis of images from at least one camera configured to acquire images of at least one moving part of the x-ray system.

The at least one set associated with the at least one trigger may comprise at least one dose reduction status parameter.

The at least one activation parameter may be pre-defined.

The at least one set may be pre-defined.

A user may associate at least one set with a pre-defined filter.

A user may associate at least one pre-defined set with a filter.

A user may associate at least one pre-defined set with a pre-defined filter.

The at least one activation parameter may be a motion status of at least one part of the x-ray system.

The at least one set may comprise at least one radiation reduction system status parameter.

The at least one radiation reduction system status parameter may comprise at least one of the following x-ray exposure parameters: (a) x-ray tube current; (b) x-ray tube voltage; and (c) Image processing.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1A is an illustration an exemplary of x-ray system in one exemplary position;

FIG. 1B is an illustration of the exemplary x-ray system of FIG. 1A in a second exemplary position;

FIG. 2A is an illustration of another exemplary x-ray system in one exemplary position;

FIG. 2B is an illustration of the exemplary x-ray system of FIG. 2A in a second exemplary position; and

FIG. 3 is an illustration of an exemplary filter.

### DETAILED DESCRIPTION OF PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

### Nomenclature

In the specifications below the following terms have the meaning described herein:
Filter: Any collimator such as typically provided in x-ray systems to limit the geometrical dimensions of the x-ray radiation including, but not limited to, 2 blades collimators, 4 blades collimators and Iris collimator whether such blades are designed to block x-ray radiation or be semi-transparent to x-ray radiation; a wedge filter as typically provided in x-ray systems; a semi-transparent filter; or any other object that interacts with an x-ray beam in order to modulate its intensity or spectral distribution.
Blade A part of the filter, typically a flat piece of material, suspended in a plane that is parallel to the detector's plane and provides the function of interaction with the x-ray beam. For example, the term “semi-transparent filter” means that the filter uses blades that are semi-transparent to x-ray. Blades can also be designed to block x-ray radiation as much as possible.
Aperture An opening in a filter allowing x-ray radiation to pass through without being filtered or blocked; or
   An area in a filter allowing x-ray radiation to pass through without interaction with blades.
X-Ray System: Any device designed to generate x-ray radiation, typically in the shape of a beam having a solid-angle, and a human or machine visible image based on the x-ray radiation.
   The term x-ray system means also an x-ray system that includes at least one filter.
   Examples of x-ray systems are Artis Zee and Ysio Max available from Siemens (Erlangen, Germany) and OEC 9900 Elite available from GE Healthcare (Waukesha, Wis., USA).
Detector A device having a surface used to capture x-ray radiation and intended to convert the spatial distribution of the x-ray radiation to another representation of this distribution, such as, but not limited to, a distribution of light in the visible range and/or a distribution of electrical charges.
   Examples of such detectors are image intensifiers such as HIDEQ 23-3 ISP Available from Siemens (Erlangen, Germany) and flat-panel-detectors such as The XRD4343RF, available from Perkin Elmer (Waltham, Mass., USA).
Computer A device used for any computation task such as, without limitations, to tasks carried by computers, processors, image processors or combinations thereof, whether built as a general-purpose computer or dedicated hardware or any combination thereof.
Trigger: Any means or method that provides an indication to deploy an action leading to a desired outcome, based on input. Such a trigger may include, but is not limited to, data representing x-ray system positioning (including but not limited to data from a motion encoder associated with a moving part of the x-ray system or from an accelerometer associated with a moving part of the x-ray system or from a camera and a computer that capture and analyses the image to provide desired information about the image) and a user enabled button.
Activation Activation of a trigger, or engagement or realization of a trigger is a situation in which the values specified for parameters associated with a trigger are realized exactly or within given tolerances or a range, when such tolerances or range are specified.
Data: The words data, information and status may be used interchangeably throughout the description.
Status: Status refers to the situation of the x-ray system as expressed in terms of any or all parameters associated with its situation. This may include, for example, without limitations, spatial orientation, distance of the detector from the x-ray source, x-ray generator operation settings, filter settings, electronic image magnification, data stored on a storage device, value of a register in the processor of the computer of the x-ray system, status of buttons operated by the user and whether it is radiating or not.
Semi: Partly, incompletely, having some of the characteristics of.
ROI Region of Interest, the area in an image that includes, but not limited to, items that are in the focus of attention of the user.
Radiation Means x-ray radiation. The term beam or x-ray beams may also be used to describe x-ray radiation.

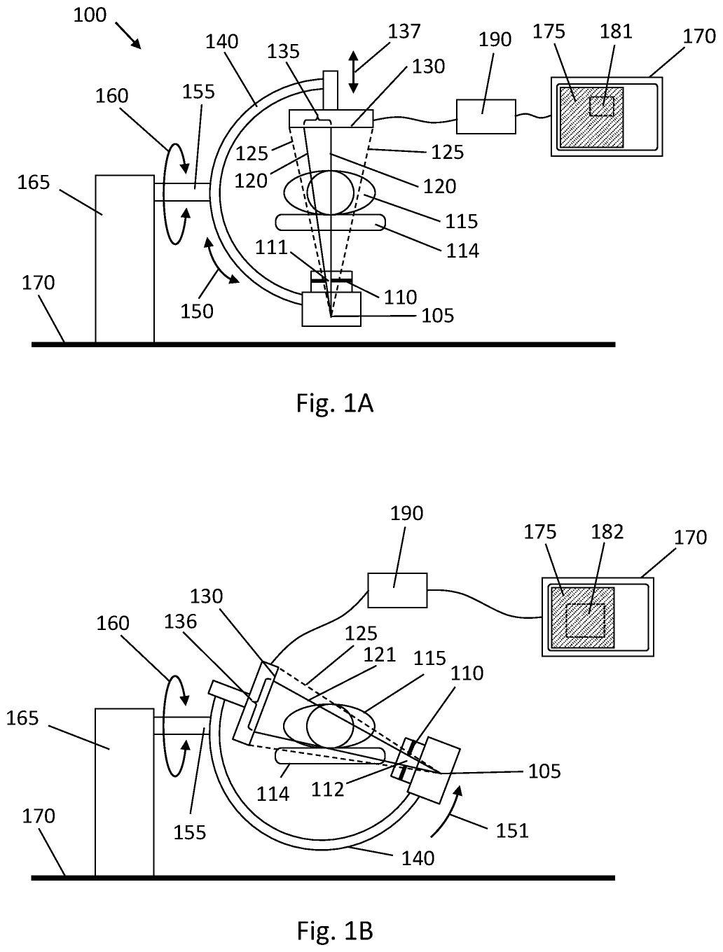

FIG. 1A and FIG. 1B illustrate one example of changes in the x-ray system imaging conditions that involve changes in imaging position and changes in the ROI and thereby in the filter settings.

Reference is made now to FIG. 1A illustrating a first example of an x-ray system 100 in a first exemplary position. X-ray source 105 is the source of x-ray radiation that is directed upward towards patient 115. The full beam angle is illustrated by two dashed lines 125. The x-ray beam is modulated by filter 110 that may be a semi-transparent filter with an aperture 111. The x-ray radiation passes through the patient table 114 and the patient 115 and arrives at detector 130. Unfiltered x-ray radiation arrives at section 135 of detector 130 and the filtered x-ray radiation arrives at the detector 130 outside 135. The x-ray radiation is then converted to a human visible image 175 on display 170. This conversion involves computer 190 that may format the image data for display, and may also process at least parts of the image, including but not limited to brightening of the filtered part of the image. Computer 190 may also include a storage device (not shown) usable for any data storing that may be required. ROI 181 is associated with section 135 of detector 130, thus, the image part inside ROI 181 is a result of an unfiltered x-ray radiation and the image part outside ROI 181 is a result of a filtered x-ray radiation.

X-ray source 105, filter 110 and detector 130 are assembled on c-arm 140 that is suspended by arm 155 and installed on pedestal 165. Pedestal 165 may be attached to floor 170.

In the example of FIG. 1A three degrees of freedom of motion are illustrated. C-arm 140 may be moved in the direction of dual head arrow 150 in order to change the directions of the x-ray radiation relative to patient 115. C-arm 140 may also be moved in the direction of dual head arrow 160 in order to change the directions of the x-ray radiation relative to patient 115 in other directions. Detector 130 may be moved in the direction of dual head arrow 137 in order to move detector 130 towards or away from x-ray source 105, determining the Source to Image Distance (SID). The x-ray system may have additional motion degrees of freedom known in the art but, without limitations, FIG. 1A illustrates only a few examples.

According to embodiments of the present invention, the x-ray system may be equipped with at least one status indicator (not shown in the drawings). Such a status indicator may provide an indication of the status of any part of the x-ray system, including but not limited to x-ray system orientation status, motion status, x-ray tube generator status, image processing parameters status, x-ray tube temperature status, etc.

FIG. 1B illustrates the x-ray system of FIG. 1A in a rotated position in direction shown by arrow 151. Due to the rotation, the direction of the x-ray beam is changed relative to patient 115. In the example of FIG. 1B, ROI 182 is also different from ROI 181 of FIG. 1A (typically because of a different anatomy view). In the example of FIG. 1B it is also shown that aperture 112 is of different size and/or position as required by the new ROI 182 and the unfiltered area of radiation arriving at detector 130 changed from 135 (FIG. 1A) to 136 (FIG. 1B).

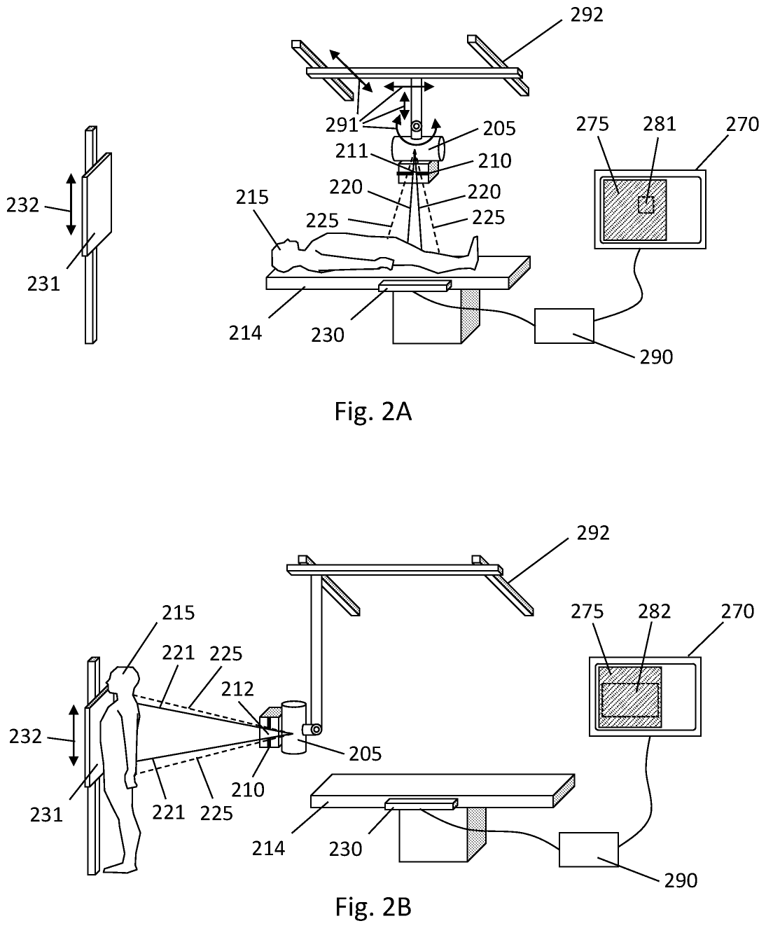

FIG. 2A and FIG. 2B illustrate a second example of changes in the x-ray system imaging conditions that involve changes in imaging position and changes in the ROI, and thereby in the filter settings.

Reference is made now to FIG. 2A illustrating a second example of an x-ray system in a first exemplary position. X-ray source 205 is the source of x-ray radiation that is directed downwards towards patient 215. The full x-ray beam angle is illustrated by two dashed lines 225. The x-ray beam illustrated by two dashed lines 225 is modulated by filter 210 that may be a semi-transparent filter with an aperture 211. The x-ray radiation passes through patient 215 and patient table 214 and arrives at detector 230. Unfiltered x-ray radiation 220 arrives at a small area of detector 230 (not shown) and the filtered x-ray radiation arrives at the area of detector 230 that extends beyond the area of beam 220. The x-ray radiation is then converted to human visible image 275 on display 270. This conversion involves computer 290 that may format the image data for display, and may also process at least parts of the image, including but not limited to brightening of the filtered part of the image. ROI 281 is associated with the small area of unfiltered radiation arriving at detector 230, thus, the image part inside ROI 281 is a result of an unfiltered x-ray radiation and the image part outside ROI 281 is a result of a filtered x-ray radiation.

X-ray source 205 and filter 210 assembly are mounted on suspension 292 that provides movements in the directions indicated by dual head arrows 291 (movement in 3-dimensional space and also rotation of x-ray source 205 and filter 210 assembly. According to embodiments of the present invention, the system may also include another detector 231 which may be moved in the directions of dual head arrow 232.

FIG. 2B illustrates the x-ray system of FIG. 2A in another position where x-ray source 205 and filter 210 assembly are moved and rotated to direct the x-ray beam towards detector 231.

In the example of FIG. 2B, ROI 282 is also different from ROI 281 (typically because of different anatomy view). In the example of FIG. 2B it is also shown that aperture 212 is of different size and/or position relative to aperture 211 of FIG. 2A as required from the new ROI 282 that is different from ROI 281 of FIG. 2A.

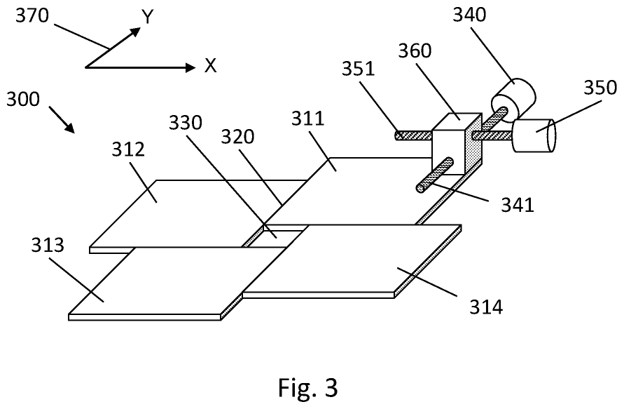

FIG. 3 shows an exemplary filter 300. There are many designs of filters in the art and FIG. 3 is provided only as an example and does not limit the scope of the present invention.

Each of blades 311, 312, 313 and 314 is typically placed in a plane that is parallel to the detector packaging surface that faces the x-ray source. The blades provide the x-ray beam filtering function. The blades are arranged in a way that provides an aperture 330 through which the x-ray beam passes without filtration.

Each of the blades is attached to a dual side nut 360 (shown only in relation to blade 311). Nut 360 is driven by two motors 340 and 350 and screws 341 and 351. This provides the motorized movement of blade 311 in two independent directions X and Y as illustrated by arrows 370.

With this mechanism each of blades 311, 312, 313 and 314 can be moved to any position in their plane in order to create any desired rectangular shape and position of aperture 330.

For a filter that is semi-transparent (semi-transparent to x-ray radiation) the blades provide the function of being semi-transparent.

It would be appreciated that since this invention relates to x-ray system that convert x-ray radiation to a human visible image, a semi-transparent filter means that the blades, being semi-transparent are designed to allow enough radiation to pass through the blades to still provide a human vision acceptable image. It would also be appreciated that typical collimators reduce radiation to a level that can not be used to provide an image thereby. Typically for systems with semi-transparent filter, 10% of the normal radiation can still be used to provide a human visible image. Below 10% transparency the human visible image may be degraded too much for practical use. As an example, 0.8 mm Cupper filter may typically be semi-transparent to pass about 10% of the radiation of x-ray system at 50 KVp (depending on the specific x-ray system).

In such an x-ray system, the image is typically processed to enhance the image using a computer 190 (FIGS. 1A, 1B) or computer 290 (FIGS. 2A and 2B). The filtered part of the image can also be processed in such computers, mainly to brighten the image that is dark due to the lower level of the filtered x-ray.

According to embodiments of the present invention, a user may specify at least one status parameter for the activation of trigger-1, assign it a value and store it for trigger-1. The user may also associate a tolerance for the at least one value, stored with trigger-1. Then, the user may store for trigger-1 a set-1 of parameters by selecting at least one status parameter and determine its value (the term “value” may also mean, without limitations, a range of values or a value with tolerances). When trigger-1 is activated (the activation values and/or the values within the tolerances are realized by the x-ray system status or the x-ray system status is within the range of activation values), the x-ray system, in response, automatically modifies the status of the x-ray system to a status where the parameters included in set-1 assume the values specified in set-1.

According to embodiments of the present invention, the user may store for trigger-1A activation parameters such as, for example, orientation parameters of the x-ray system. For a vertical position example as shown in FIG. 1A the user may store for trigger-1A the position of the x-ray system in terms of rotation 150, rotation 160 and SID 137. Each of these parameters assumes a value representing this vertical position. Part or all of these values may also assume a tolerance value or a range stored for trigger-1A.

The user may store filter setting values with set-1A that is associated with trigger-1A, such as an aperture 111 first size and/or position which corresponds with ROI 181 of FIG. 1A.

The user may also store activation parameters for trigger-1B such as orientation parameters of the x-ray system as shown in the exemplary position of FIG. 1B and may store also tolerances for one or more parameters or ranges for one or more parameters. Other filter values may be stored with set-1B that is associated with trigger-1B, such as values that provide the unfiltered image part of 136 (and 182) of FIG. 1B.

The user may assign filter setting values for set-1B that is associated with trigger-1B such as an aperture 112 of a second size and/or position which corresponds now with ROI 182 of FIG. 1B.

According to embodiments of the present invention, when the relevant values of x-ray system status parameters are the same as the activation values of a trigger or are the same within the assigned tolerance or are within the specified range of values, the status parameter of the x-ray system are then considered as satisfying the activation values of the trigger. When this happens, it is said that the status of the x-ray system satisfies the activation values of trigger. The trigger then will be activated and the system status will change so that the values parameters included in the set associated with the filter will coincide with any of the values, the values within assigned tolerances or the range of values of the same parameter included in the set.

As a result, when the x-ray system is positioned at or near the position of FIG. 1A, and the position status of the x-ray system satisfies the activation values of trigger-1A, the x-ray system automatically sets filter 110 to the position values of set-1A that provide aperture 111 and, as a result, unfiltered area 135 that fits ROI 181 of FIG. 1A.

If the x-ray system is positioned at or near the position of FIG. 1B so that the x-ray system position status satisfies the activation values of trigger-1B, the x-ray system automatically sets filter 110 to the position values of set-1B that provide aperture 112 and, as a result, unfiltered area 136 that fits ROI 182 of FIG. 1B.

It would be appreciated that a trigger based on the x-ray system position status is provided as an example only and the trigger may be based on any status parameters. Also, setting filter 110 in response to trigger activation is provided as an example and the status of any of filter 110 and/or other module can be set as a response to trigger activation.

It would be appreciated that the same example can be realized for other x-ray systems such as the x-ray system example of FIG. 2A and FIG. 2B. Trigger-2A is intended, for example, to include the position values of the x-ray system of FIG. 2A for triggering purpose, possibly with tolerances. Set-2A is associated with trigger-2A to may include values for filter 210 settings, in this example: aperture 211 data. Set-2A may also or alternatively include, without limitations, x-ray exposure parameters (voltage typically in KVp units and current typically in mA units, both applied by the generator to the x-ray tube) and/or image processing parameters that are optimized to, for example, human torso anteroposterior imaging.

Trigger-2B is intended to include the position values of the x-ray system of FIG. 2B, possibly with tolerances. Set-2B is associated with trigger-2B to include also values for filter 210 settings, in this example: aperture 212 data. Set-2B may also or alternatively include, without limitations, x-ray exposure parameters (voltage typically in KVp units and current typically in mA units and/or image processing parameters) that are optimized to, for example, human torso lateral imaging. When moving the x-ray system between the positions of FIG. 2A and FIG. 2B, trigger-2A and trigger-2B are realized based on the x-ray system position status and the x-ray system sets its status to realize the values of set-2A (aperture 211 and maybe other parameters such as x-ray exposure parameters and/or image processing parameters) or set-2B (aperture 212 and maybe other parameters such as x-ray exposure parameters and/or image processing parameters) accordingly.

It would be appreciated that a trigger based on the x-ray system position status is provided as an example only and the trigger may be based on any associated status parameters.

An x-ray system may include more than one filter. For example, an x-ray system may include a semi-transparent filter and also an opaque filter. A stored set may include setting parameters for both these filters.

In another example of a set associated with a trigger, the set may include parameters that activates a semi-transparent filter to filter a part of the x-ray radiation and also activates image processing in computer 290 to make the filtered part of the image brighter or modify it in any other way. In another set associated with another trigger the associated set may set computer 290 to not process the filtered part of the image.

In yet another embodiment of the present invention a set associated with a trigger may include position parameters of an x-ray system such as rotation 150, rotation 160 and sensor position in direction 137 of FIG. 1A or 291 of FIG. 2A. The set may be associates with a trigger that is also associated with any activation parameters. For example, the trigger activation parameter may comprise an alpha-numeric string. When this string is entered to the x-ray system by the user or by any other means, the x-ray system moves and positions itself according to the position values of the associated set. Such motion can be executed using, for example, motors such as in Artis Zee available from Siemens (Erlangen, Germany).

In yet another embodiment of the present invention a set associated with a filter may include position parameters of an x-ray system such as rotation 150, rotation 160 and sensor position in direction 137 of FIG. 1A or 291 of FIG. 2A and also filter settings.

In another exemplary embodiment of the present invention, activation parameters of the trigger may consist of position or motion detection of any part of the x-ray system, including for example moving of patient table 114 of FIG. 1A and 214 of FIG. 2A.

Position or motion detection may be done in a variety of ways including but not limited to:
1. Reading position values of at least one encoder of the x-ray system mechanical structure that indicates movement of at least a part of the x-ray system through changes in values read from such at least one encoder;
2. Status indication of at least one control that provides the user the ability to activate at least one motor that moves at least one part of the x-ray system. The motion information is deduced from an indication that a control of motion has been engaged.
3. Readings values from at least one accelerometer attached to a movable part of the x-ray system and drawing information regarding movements and/or position of the movable part using any method, such one example is methods used for inertial navigation systems such as described in “Strapdown Inertial Navigation systems: An Algorithm for Attitude and Navigation Computation”, by R. B. Miller, Aeronautical Research Laboratories Melbourne, Australia.
4. Image analysis during radiation can be used to extract motion indication from the images produced from the x-ray radiation. For example, displacement direction and speed can be extracted by estimation of cross-correlation between successive images. Pattern recognition, perspective changes and parallax between patterns can provide additional motion information, including rotation of the imaging part around the patient. Such estimation can be done using computer 190/290. Techniques for determination of camera position can be implemented for x-ray imaging system (that is in fact a camera of special characteristics) are described, for example, in “Analysis of determining camera position via Karhunen-Loeve transform”, P. Quick and D. Capson, published by IEEE, ISBN number: 0-7695-0595-3. When applying such algorithms for x-ray camera, the x-ray imaging system should be treated as a virtual camera located at the focal spot and directed towards the object under examination. Fiducials visible in x-ray imaging, such as made from lead, can be attached, in a known geometry, to the table and be used for image analysis to extract position status information of the imaging system and the table relative to each other. Such analysis can be carried out using known methods such as “Analysis of determining camera position via Karhunen-Loeve transform”, P. Quick and D. Capson, published by IEEE, ISBN number: 0-7695-0595-3.
5. A video camera may be attached to a movable part of the x-ray system. Successive images from the camera may be analyzed to extract information about the x-ray system motion. The analysis can be made by computer 190 using cross-correlation, pattern recognition, perspective changes and parallax between patterns. Techniques for determination of camera position are described, for example, in “Analysis of determining camera position via Karhunen-Loeve transform”, P. Quick and D. Capson, published by IEEE, ISBN number: 0-7695-0595-3.
6. A video camera that captures at least a part of the x-ray system structure. Successive images from the camera may be analyzed to extract information about the x-ray system motion. The analysis can be made by computer 190 using cross-correlation, pattern recognition, perspective changes and parallax between patterns. Techniques for determination of camera position are described, for example, in “Analysis of determining camera position via Karhunen-Loeve transform”, P. Quick and D. Capson, published by IEEE, ISBN number: 0-7695-0595-3.
7. Any combination of the above techniques for providing motion indication. Motion indication trigger can be associated with a set that has benefits associated with the motion. According to embodiments of the present invention, the set (associated with the trigger that includes motion parameters) may include x-ray system parameters that are associated with x-ray radiation exposure, such parameter values are directed towards reduction of x-ray radiation during motion. This may be useful for users who do not need full standard image quality during motion because, for example, the motion is intended for patient and system positioning and requires only general anatomy imaging, without a need for high resolution details. Such parameters may include, but are not limited to:
1. X-ray tube current (mA) reduction relative to typical imaging parameters;
2. X-ray tube voltage (KVp) change relative to typical imaging parameters;
3. Introducing a semitransparent filter over all or a part of the image to reduce radiation;
4. Any combination of the above.

Such adjustments can be enhanced with a complementary automatic brightness adjustment of the image using computer 190/290 to compensate at least partially for the darker image resulting from reduced x-ray radiation.

Any other trigger may be used to engage radiation reduction parameters. One additional example for such a trigger is a trigger that is activated by a specific setting of a filter. For example, a fully open filter may activate a trigger to reduce radiation as described above.

A value of a status parameter in a set may also include a tolerance or a range that may be specified also by the user. In this case, after a trigger is activated, the x-ray system automatically changes its status to realize a new status in which the actual value of the parameter is the same as the value of the set within the specified tolerance.

As described above, the user can define and store a trigger that contains at least one status parameter, with a value assigned to this at least one parameter and may also specify a tolerance to this value. It is also described above that the user can define and store a set associated with the filter, this set contains at least one status parameter, with a value assigned to this at least one parameter and may also specify a tolerance or ranges to this value.

In another exemplary embodiment of the present invention, at least one trigger may be pre-defined in the x-ray system and the user defines and stores only the set associated with this filter. In another exemplary embodiment of the present invention, at least one set may be pre-defined in the x-ray system and the user defines and stores only the trigger to which this set will be associated. In another exemplary embodiment of the present invention, at least one trigger is pre-defined and at least one set is predefined and the user may define which of said at least trigger and which of said at least one set are associated with each other. Pre-defining of any of a trigger or a set may be performed by any person such as the manufacturer, by the user or by a service engineer.

More than one existing set can be associated with a single trigger. Such association can be defined by a user so that the user associates to a trigger more than one existing set.

It would be appreciated that a trigger may also include an electrical switch, a mechanical switch or a software switch presented to the user on a display GUI. Activating or toggling of the switch provides the trigger and the x-ray system changes its status automatically in accordance with the values of the parameters stored with the set associated with this trigger.

It would also be appreciated and emphasized that a trigger may contain values of any x-ray system status parameters and also a set may contain values of any x-ray system status parameters and the examples above do not limit triggers and sets content.

It would be appreciated by those skilled in the art that the above described methods and technologies are not limited to the configurations and methods mentioned herein above as examples. These are provided as examples and other configurations and methods can be used to optimize final result, depending on the specific design and the set of technologies implemented in association with the x-ray.

It would be appreciated by those skilled in the art that the above embodiments are described in a way of example only and do not specify a limited scope of the invention.

Rather the scope of the present invention is defined by the appended claims and includes combinations and sub-combinations of the various features described hereinabove as well as variations and modifications thereof which would occur to persons skilled in the art upon reading the foregoing description.

1. An x-ray system comprising:
an x-ray source;
a detector;
a table;
at least one moving part configured to at least one of:
a. position said x-ray source relative to a patient;
b. position said detector relative to a patient; and
c. position said table;
at least one system position status value associated with at least one system position status of said at least one moving part;
a filter;
means for automatically setting said filter according to said at least one system position status;
said means for automatically setting said filter according to said at least one system position status comprises:
at least one trigger; and
at least one activation parameter associated with said at least one trigger;
said at least one activation parameter is selected from the group consisting of:
(a) at least one system position status value;
(b) at least one system position status value with a tolerance; and
(c) at least one range of system position status values;
at least one set associated with said at least one trigger, said at least one set comprises at least one filter status parameter, said at least one filter status parameter comprises at least one of:
(1) a filter status value of said filter;
(2) a filter status value with a tolerance; and
(3) a range of filter status values; and
wherein said filter status is changed according to said set upon activation of said at least one trigger.

2. The system of claim 1, wherein a user can configure at least one of:
(a) at least one activation parameter of said at least one trigger; and
(b) at least one filter status parameter of said at least one set.

3. The system of claim 1, wherein said at least one system position status value is provided from at least one of the group consisting of:
(a) at least one accelerometer connected with at least one moving part of said x-ray system;
(b) at least one encoder indicating position of at least one moving part of said x-ray system;
(c) a camera;
(d) analysis of at least one x-ray image;
(e) analysis of at least one x-ray image comprising fiducials of known geometry;
(f) analysis of images from at least one camera attached to at least one moving part of said x-ray system; and
(g) analysis of images from at least one camera configured to acquire images of at least one moving part of said x-ray system.

4. The system of claim 1, further configured to reduce radiation during motion of at least one of said at least one moving part.

5. The system of claim 4, wherein said radiation reduction is performed according to one of said trigger and a second trigger.

6. The system of claim 4, wherein a user can configure at least one of:
(a) an activation motion parameter of said at least one trigger; and
(b) at least one radiation reduction parameter included in said at least one set associated with said at least one trigger.

7. The system of claim 4, wherein motion status information is provided from at least one of the group consisting of:
(a) at least one accelerometer connected to said at least one moving part;
(b) at least one encoder indicating position of at least one of said at least one moving parts;
(c) a camera;
(d) x-ray image analysis;
(e) analyzing x-ray image that includes fiducials of known geometry; and
(f) analyzing images from at least one camera attached to at least one of said moving parts; and
(g) analyzing images from at least one camera configured to acquire images of at least one of said moving part.

8. The system of claim 4, further comprising means for enhancing at least one image obtained with said reduced radiation.

9. The system of claim 8, wherein said enhancing at least one image obtained with said reduced radiation comprises increasing brightness of at least a part of said image.

10. An x-ray system comprising:
at least two system statuses;
at least one system status value associated with at least one of said at least two system statuses;
at least one trigger;
at least one activation parameter associated with said at least one trigger;
said at least one activation parameter is selected from the group consisting of:
(a) at least one system status value;
(b) at least one system status value with a tolerance; and
(c) at least one range of system status values;
at least one set associated with said at least one trigger, said at least one set comprises at least one system status parameter, said at least one system status parameter comprises at least one of:
(1) a system status value;
(2) a system status value with a tolerance; and
(3) a range of system status values; and
wherein said system status is changed according to said set upon activation of said at least one trigger.

11. The system of claim 10, wherein said at least one activation parameter associated with said trigger is specified by a user.

12. The system of claim 10, wherein said at least one system status parameter of said at least one set is specified by a user.

13. The system of claim 10, wherein said at least one system status parameter of said at least one set is a status parameter of a filter.

14. The system of claim 10, wherein said at least one set comprises at least one radiation reduction status parameter.

15. The system of claim 10, wherein said at least one set comprises at least one x-ray tube voltage parameter.

16. The system of claim 10, wherein said at least one set comprises at least one x-ray tube current parameter.

17. The system of claim 10, wherein said at least one set comprises at least one x-ray system position status parameter.

18. The system of claim 17, wherein said x-ray system is configured to automatically move to said at least one x-ray system position of said at least one set upon activation of said at least one trigger.

19. The system of claim 10, wherein at least one datum of at least one of said at least two system statuses is provided from at least one of the group consisting of:
(a) at least one accelerometer connected with at least one moving part of said x-ray system;
(b) at least one encoder indicating position of at least one moving part of said x-ray system;
(c) a camera;
(d) analysis of at least one x-ray image;
(e) analysis of at least one x-ray image comprising fiducials of known geometry;
(f) analysis of images from at least one camera attached to at least one moving part of said x-ray system; and
(g) analysis of images from at least one camera configured to acquire images of at least one moving part of said x-ray system.

20. The system of claim 10, wherein said at least one trigger is selected from the group consisting of:
(1) a mechanical switch;
(2) an electrical switch;
(3) a user interface graphical switch displayed by a computer on a monitor.

21. The system of claim 10, wherein said at least one set comprises at least one parameter related to image processing.

22. The system of claim 10, wherein said at least one activation parameter comprises at least one alpha-numeric string.

23. The system of claim 10, wherein said at least one activation parameter comprises at least one motion system status.

24. The system of claim 23, wherein at least one datum of said at least one motion system status is provided from at least one of the group consisting of:
(a) at least one accelerometer connected with at least one moving part of said x-ray system;
(b) at least one encoder indicating position of at least one moving part of said x-ray system;
(c) a camera;
(d) analysis of at least one x-ray image;
(e) analysis of at least one x-ray image comprising fiducials of known geometry;
(f) analysis of images from at least one camera attached to at least one moving part of said x-ray system; and
(g) analysis of images from at least one camera configured to acquire images of at least one moving part of said x-ray system.

25. The system of claim 23, wherein said at least one set associated with said at least one trigger comprises at least one dose reduction status parameter.

26. The system of claim 10, wherein said at least one activation parameter is pre-defined.

27. The system of claim 10, wherein said at least one set is pre-defined.

28. The system of claim 10, wherein a user associates at least one set with a pre-defined filter.

29. The system of claim 10, wherein a user associates at least one pre-defined set with a filter.

30. The system of claim 10, wherein a user associates at least one pre-defined set with a pre-defined filter.

31. The system of claim 10, wherein said at least one activation parameter is a motion status of at least one part of said x-ray system.

32. The system of claim 10, wherein said at least one set comprises at least one radiation reduction system status parameter.

33. The system of claim 32, wherein said at least one radiation reduction system status parameter comprises at least one of the following x-ray exposure parameters:
(a) x-ray tube current;
(b) x-ray tube voltage; and
(c) Image processing.
